(19) United States
(12) Patent Application Publication
(10) Pub. No.: US 2007/0282154 A1
(43) Pub. Date: Dec. 6, 2007

(54) Device For Brachytherapy With at Least One Radiation Source For Introduction Into the Body

(76) Inventor: Anwer Puthawala, Buckenhof (DE)

Correspondence Address: JORDAN AND HAMBURG LLP, 122 EAST 42ND STREET, SUITE 4000, NEW YORK, NY, 10168, US

(21) Appl. No.: 11/662,212

(22) PCT Filed: Sep. 9, 2005
(86) PCT No.: PCT/EP05/09690

§ 371 Date: May 21, 2007

(30) Foreign Application Priority Data
Sep. 10, 2004 (DE) 10 2004 043 878.1

(51) Int. Cl. A61N 5/10

(52) U.S. Cl. 600/3

(57) ABSTRACT

A device for brachytherapy, has at least one radiation source which can be introduced into the body and which is arranged at the tip of a flexible guide cable and which is easy and safe to operate. The guide cable is arranged so that it can be wound and unwound in a helical groove of a rotatable reel element and is guided from there to an outlet via a shield, from which outlet the introduction of the radiation source is effected with a catheter. The rotation of the reel element and thus the movement of the radiation source are locked.

DE 197 58 234 C2 describes a device for treating stenoses in blood vessels, said device being provided with a flexible guide cable which can be introduced into a blood vessel and which has a radioactive radiation source at its tip. The operator pushes the cable by hand into the blood vessel, under X-ray control, until the radiation source lies in the area of the stenosis, where the tissue narrowing the cross section of the blood vessel is destroyed by the radiation. Manipulating the cable without guide means requires great dexterity and care on the part of the operator.

The object of the invention is to make available a device of the type mentioned at the outset with which it is possible to operate more easily and more safely than in the prior art.

According to the invention, this object is achieved by the features of patent claim 1. The device according to the invention permits a safe movement and guidance of the guide cable, by which means, the path traveled by the radiation source can be fixed exactly.

A particularly expedient embodiment of the device according to the invention, in which the radiation source cannot be moved beyond defined end positions, is set out in claim 7.

The invention is explained in more detail below with reference to an illustrative embodiment depicted in the drawing, in which:

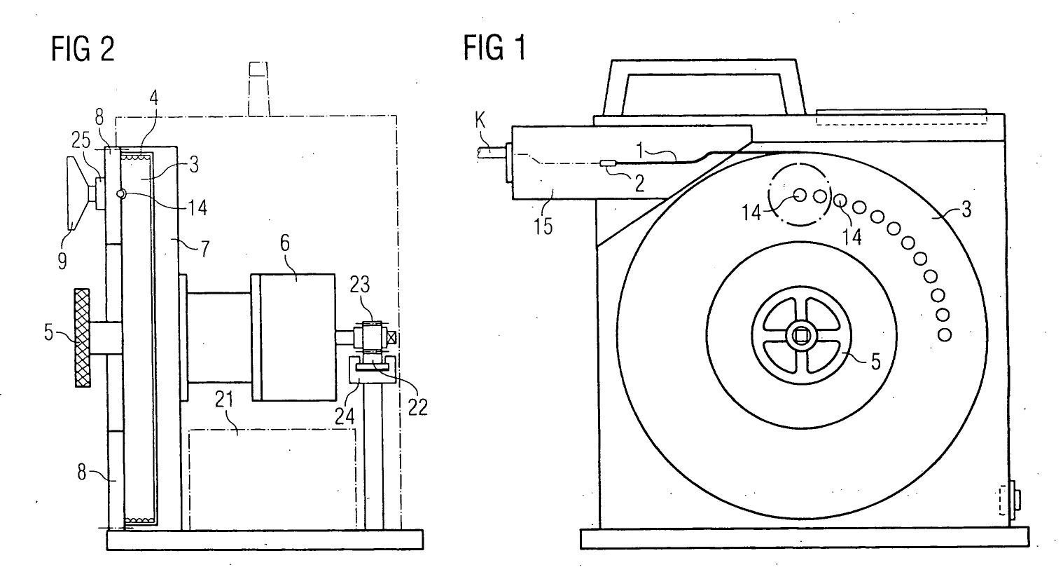

FIGS. 1 and 2 show views of a device according to the invention from two directions perpendicular to one another, and

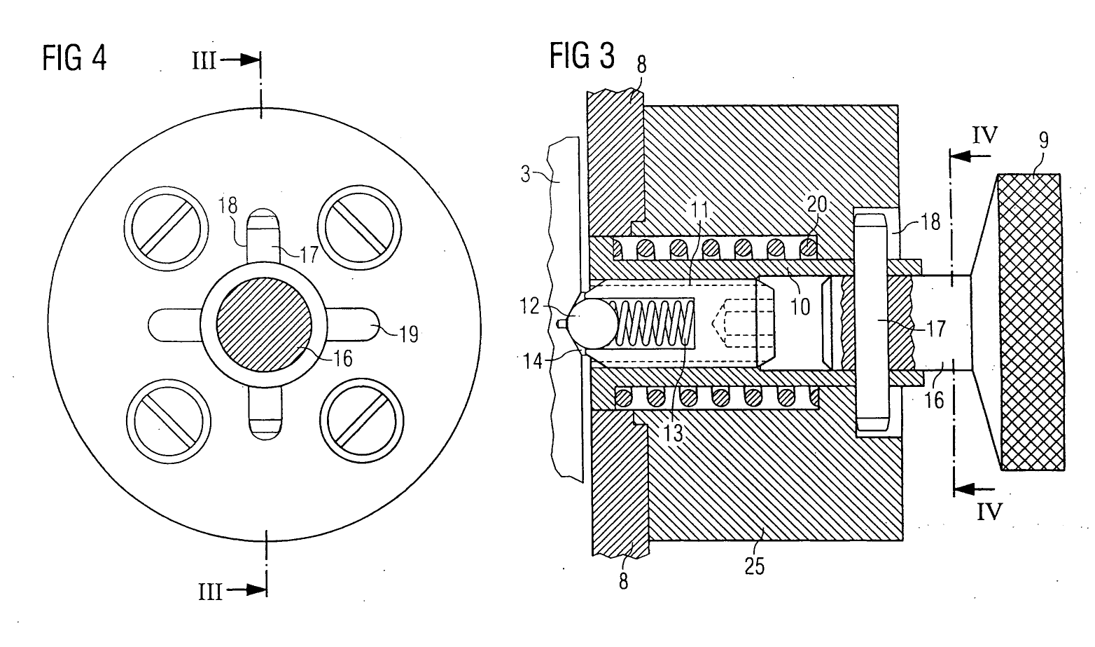

FIGS. 3 and 4 show two cross sections IV and III of the locking arrangement in the device according to FIGS. 1 and 2.

In FIGS. 1 and 2, a flexible guide cable 1 is shown with a radioactive radiation source 2 applied at its tip. The guide cable 1 can be wound and unwound on a rotatable reel element 3. It lies in a helical groove 4 in the reel element 3. A handwheel 5 permits rotation of the reel element 3. Alternatively, the reel element 3 can also be turned by a motor 6.

The reel element 3 is mounted rotatably in a housing 7, to which a ring 8 is connected. The ring 8 is in a fixed position and supports a locking arrangement which, according to FIGS. 2 and 3, has a handgrip 9 that can be turned in steps of 90 degrees. Connected to the handgrip 9 there is a sleeve 10 in the interior of which a further sleeve 11 is screwed, and in which sleeve 11 a locking ball 12 can be moved longitudinally counter to the force of a spring 13. The locking ball 12 engages in depressions 14 on the surface of the reel element 3.

In the rest state, the radiation source 2 lies in a shield 15, e.g. of tungsten. The guide cable 1 is guided with a kink formation in a channel in the shield 15 so that the radiation of the radiation source 2 cannot escape from the shield 15.

To treat a stenosis, the reel element 3 is turned counterclockwise by hand or by motor (FIG. 1) so that the radiation source 2 emerges on the left from the shield 15 and, through further rotation of the reel element 3, can be introduced via a plastic catheter K into a blood vessel. The advance movement in the blood vessel takes place easily and safely until the radiation source 2 reaches the stenosis. The catheter K is applied at the outlet of the shield 15, e.g. by means of a quick-coupling.

After the radiation exposure time has elapsed, the reel element 3 is turned clockwise and the radiation source 2 is thus withdrawn from the blood vessel.

The movement of the radiation source 2 in the blood vessel can take place in predetermined steps. The step length is defined by the distance between the depressions 14. The locking arrangement shown in FIGS. 3 and 4 determines the step length.

The locking arrangement can be released in order to permit a continuous movement of the source. For this purpose, a pin 17 is inserted through the shaft 16 of the handgrip 9 and engages in grooves 18, 19 offset by 90 degrees in a holder 25 connected to the ring 8. The groove 18 is deeper than the groove 19. In the position of the pin 17 illustrated, a spring 20 presses the sleeve 10 against the reel element 3 and the ball 12 into the depressions 14. If the handgrip 9 is turned through 90 degrees, the pin 17 lies in the shallower groove 19 and the sleeve 11 with the ball 12 is withdrawn from the respective depression 14. The handgrip 9 can be also be turned electromagnetically or by motor and can be adjusted in the axial direction.

It is thus possible for the radiation source 2 in the blood vessel to be moved either stepwise, with locking, or continuously. If the radiation source 2 is moved with locking, the operator has to apply more force than in the case of non-locked movement to turn the handwheel 5 so as to move from one locked position to another desired locked position. The same applies by analogy to an electromagnetic or motor movement.

FIG. 2 also shows the control electronics 21 and a toothed rack 22 for limiting the end position of the rotation movement of the reel element 3 and thus the radiation source 2. The toothed rack 22 is moved perpendicular to the plane of the drawing in a bar 24 by means of a pinion 23 on the shaft of the motor 6 and of the handwheel 5 upon rotation of this shaft. Adjustable limit stops (not shown) limit the path of the toothed rack 22 and thus of the reel element 3.

When the radiation source 2 is located in the shield 15, this rest position is secured by the locking ball 12 locking in one of the depressions 14. In this way, the radiation source 2 cannot be withdrawn from the shield 15 as a result of inadvertent activation of the handwheel 5.

In addition to its use for treating stenoses in blood vessels, the device according to the invention can also be used to treat tumors, by introducing the radiation source 2 into a tumor.

List of Reference Labels

1 guide cable

2 radiation source

3 reel element

4 groove

5 handwheel

6 motor

7 housing

8 ring

9 handgrip

10 sleeve

11 sleeve

12 locking ball

13 spring

14 depression

15 shield

16 shaft

17 pin

18 groove

19 groove

20 spring

21 control electronics

22 toothed rack

23 pinion

24 bar

K catheter

1. A device for brachytherapy with at least one radiation source which can be introduced into the body and which is arranged at the tip of a guide cable, said guide cable being arranged so as to be wound and unwound on a reel element and being guided from the reel element to an outlet via a shield, from which outlet the introduction of the radiation source into the body is effected via a catheter that can be applied on the shield, and in which the rotation movement of the reel element is locked in predetermined steps in such a way that a stepwise rotation takes place under application of a force for providing a locking position.

2. The device as claimed in claim 1, in which the guide cable lies in a helical groove in the reel element.

3. The device as claimed in claim 1, in which the guide cable is guided with a kink formation in the shield between the reel element and the outlet.

4. The device as claimed in claim 1, in which the reel element is mounted so as to rotate.

5. The device as claimed in claim 4, in which the reel element can be rotated via a handwheel.

6. The device as claimed in claim 1, in which the rotational movement of the reel element is locked by a releasable lock.

7. The device as claimed in claim 1, in which means are provided for adjustable limiting of the end position for the rotation movement of the reel element.
